(19) United States
(12) Patent Application Publication
(10) Pub. No.: US 2025/0040915 A1
(43) Pub. Date: Feb. 6, 2025

(54) MEDICAL SAMPLING DEVICE FOR CAPTURING BIOANALYTES

(71) Applicant: IDRIS ONCOLOGY B.V., LEIDEN (NL)

(72) Inventors: Hans Peter MULDER, Leiden (NL); Peter Immanuel WIEGMAN, Leiden (NL)

(21) Appl. No.: 18/719,685

(22) PCT Filed: Dec. 16, 2022
(86) PCT No.: PCT/EP2022/086463

§ 371 Date: Jun. 13, 2024

(30) Foreign Application Priority Data
Dec. 16, 2021 (NL) 2030156

(51) Int. Cl. A61B 10/00

(52) U.S. Cl. CPC A61B 10/0045

(57) ABSTRACT

The invention provides a medical sampling device for capturing bioanalytes, including:
a wire having a receptor part, the receptor part having receptors to bind target bioanalytes,
a channel device defining a channel to receive the wire, the channel having a proximal end and a distal end,
wherein the wire is movable with respect to the channel between at least an extended position in which at least an intravenous section of the receptor part extends from the distal end of the channel and a retracted position in which the intravenous section of the receptor part is retracted into the channel,
wherein the channel device includes a liquid inlet arranged proximally with respect to at least a part of the intravenous section of the receptor part when the wire is arranged in the retracted position.

### TECHNICAL FIELD

The present invention relates to a medical sampling device for capturing bioanalytes such as bacteria, extracellular vesicles, circulating tumor cells (CTCs) and the like, for subsequent analysis.

### BACKGROUND

Interest in extracellular vesicles (EVs) as potential circulating biomarkers has grown rapidly in recent years. EVs are released from virtually all cell types, including disease associated cells such as tumour cells, into the extracellular space. Their contents, derived from a parent cell, consists of a multitude of different proteins and nucleic acids. EVs can be isolated from human biofluids including blood, urine and cerebrospinal fluid, making them ideal candidates for diagnostic and prognostic analysis. However, this has yet to be implemented within a clinical setting due to low concentrations of EVs in comparison to other serum constituents, such as albumin and apolipoproteins.

Traditionally, ultracentrifugation was the predominant method used to isolate EVs. Commercially available reagent kits such as miRCURY, ExoQuick and Invitrogen Total Exosome Isolation Reagent can be used to obtain EV pellets using centrifugation alone, however impurities are often present. Currently, size exclusion chromatography (SEC) is becoming the more popular method for EV isolation. There are several SEC based EV isolation kits commercially available, including the ExoQuick Kit (System Biosciences) and qEV (Izon). They require laboratory expertise, impurities are often present and they cannot isolate specific EV subsets such as tumour-derived EVs.

More novel EV isolation techniques are in development but have yet to reach the market. In a prior art isolation technique aqueous poly-ethylene Glycol (PEG) is used that facilitates the formation of EV aggregates that can be isolated via centrifugation. However, contamination of soluble non EV material was significant and resulted in a loss of purity. Immunoaffinity capture of EVs has been adopted in multiple studies. For example, processed blood samples can be incubated with magnetic beads or gold loaded ferric oxide nanocubes that have been coated with antibodies that have affinity to EV-specific tetraspanins (CD9, CD63, CD81). Other methods have used markers such as epithelial cell adhesion molecule (EpCAM) to immunoaffinity enrich EVs. Immunoaffinity enrichment requires a significantly large sample volume, particularly when isolating subsets such as tumour-derived EVs.

Multiple microfluidic devices have been developed for EV isolation, segregating them based on immunoaffinity, size and density. While promising, microfluidic devices are very expensive to manufacture and require specialised equipment. Furthermore, they operate at low throughput and are generally difficult to scale in production.

US 2020/0103414A1 discloses a medical device that has been developed to isolate and remove EV or viral particles from a patient's blood (apheresis). The device attaches to a dialysis machine through which the patient's blood is filtered. EVs exit the blood through a porous membrane where they come into contact with an affinity matrix composed of affinity capture agents (antibodies, antigens, proteins or aptamers). As this device requires specialist dialysis equipment, it severely limits where it can be used in a point of care setting.

A recent global report by the WHO on the epidemiology and burden of sepsis states that 1 in 5 deaths worldwide are due to sepsis. The definition of sepsis, and criteria for its diagnosis, have changed in recent years as we continue to further understand the mechanisms of the disease. Traditionally patients had to be diagnosed with systemic inflammatory response syndrome (SIRS) to be diagnosed with sepsis. Any two of the following criteria were needed for SIRS diagnosis: body temperature >38° C. or <36° C., heart rate >90, respiratory rate >20 or pCO2<32 mm Hg, WBC >12,000 or <4000 or >10% bands.

A diagnosis of sepsis had to include a prior diagnosis or SIRS, plus clinical evidence of infection (bacteremia, infiltrate on chest Xray, abscess on imaging study). The Journal of the American Medical Association recently published a proposal, sepsis-3, redefining and updating previous definitions of sepsis. Currently sepsis is defined as “life-threatening organ dysfunction, caused by a dysregulated host immune response to infection”. Organ dysfunction is most commonly assessed using the sequential organ failure assessment score (SOFA).

There is no gold standard to diagnose sepsis. To diagnose bacteraemia, a common cause of sepsis, blood cultures are generally required before administration of antibiotics which can take up to several days. This is highly inefficient, as sepsis diagnosis in the early stages, followed by quick therapeutic interventions, drastically reduces mortality. Studies have shown that each one-hour delay in diagnosis can increase the risk of mortality by 7-12%.

The urgent need to start anti-bacterial therapeutic interventions, coupled with a lack of efficient bacteraemia diagnostic technology, often results in administration of broad-spectrum antimicrobial therapies, increasing the risk of patient harm and a rise in pathogen antimicrobial resistance. It is vital that new technology is developed to quickly detect bacteria within a patient's blood, ideally in a point of care setting. The WHO (2020) has called on the global community to “develop rapid, affordable and appropriate diagnostic tools, particularly for primary and secondary levels of care, to improve sepsis identification, surveillance, prevention and treatment”.

As bacteria are difficult to immediately detect from a blood sample, due to the low number of bacteria in the blood of most bacteremic patients, assays have been developed for sepsis diagnosis that detect blood plasma proteins that have undergone changes. Neutrophil expression of CD64 (activation marker) has been shown to increase under septic conditions. Multiple POC devices have been developed to detect changes in neutrophil CD64 expression.

Microfluidic POC devices have been developed that capture CD64+ cells from whole blood via affinity. The number of CD64+ neutrophils captured correlates with the likelihood of a patient having sepsis. Other known devices are capable of measuring the number of activated circulating leukocytes based on the cell's electrical signature in response to alternating current (dielectrophoresis) to determine the likelihood of a patient having bacterial sepsis. Further devices have been developed that aim to diagnose sepsis based on blood lactate or procalcitonin levels. None of these types of devices can accurately diagnose bacteraemia sepsis with the specificity, accuracy and timeliness required to be used in a clinical point of care setting.

Pathogen isolation, specifically of bacteria, coupled with downstream analysis (e.g., on-chip DNA extraction) would be the ideal mechanism to diagnose bacterial sepsis. Lab-on-chip microfluidic based devices have been developed that can separate bacteria from whole blood and subsequently lyse them via chemical or mechanical means to allow for DNA extraction and downstream analysis (predominantly PCR). While promising, these devices have yet to demonstrate bacterial isolation from blood that has low circulating concentrations of bacteria (they were developed using blood spiked with very high concentrations of bacteria (10⁶ CFU/mL, not representative of sepsis), and they can only sample low volumes of blood (around 8 ml). Furthermore, the risks of contaminations of blood samples are still high.

Extracorporeal devices that can cleanse the blood of bacteria have been developed. These devices are used as a therapeutic tool to remove circulating bacteria from the blood. A device is developed, similar to a dialysis machine, that can sample up to 1.5 L/h of whole blood in vitro which is then returned to the body. The device removes bacteria via flowing the blood over magnetic nanobeads coated with a binding lectin that can capture multiple different pathogens. While having a relatively high flow sample rate, this device and others, require removal of whole blood from the body. This significantly increases the risk of coagulation and infection. Furthermore, they require extracorporeal equipment with filtration in a hospital and are therefore unsuitable for a low-cost point of care diagnostic device.

The aforementioned devices have yet to be made commercially available. The only FDA cleared, point of care device on the market that requires no complex instrumentation is the EPOC blood analysis system. It diagnoses sepsis via blood gas analysis to determine a patient's SOFA score. It is portable and can give a read out with a 92 μL blood sample. However, it cannot detect bacteria in the blood and thus cannot diagnose bacteraemia.

CN107625524 discloses a PICC/CVC assembly and a circulating tumor cell in-body capturing apparatus are provided. The capturing apparatus comprises an elongated capturing body, a joint connected to the tail end of the elongated capturing body and a connection tube disposed in the joint and having a valve. When circulating tumor cell detection is required, an existing PICC/CVC heparin cap is screwed off and the elongated capturing device of the capturing apparatus is disposed in a channel formed by an existing PICC/CVC conduit, so the capturing body can contact with blood of a human body blood vessel.

US 2013/0261373 discloses a system for magnetic attraction of objects. In an embodiment, the system comprises a magnetic function element guided through a cannula. The cannula may be inserted into a blood vessel through a vascular access. The magnetic function element may be used to convey magnetic particles and magnetically labelled cells to a desired site of action.

US 2005/0276727 discloses a device for measuring or identifying one or more component of interest from liquid samples arranged in a plurality of wells in a multiwell plate. In an embodiment, the device can be used in combination with a medical catheter passing through the skin and vein wall of a patient to position the device inside a vein with blood flow. In this position, the device has been fully depressed through catheter so that the extraction phase is exposed to flowing blood outside of catheter.

WO2005/062940 discloses a biological surveillance probe for detecting disease comprising an elongated body and a binding surface attached to the elongated body. At least one binding partner is attached to the binding surface to bind at least one complementary target. The elongated body may be introduced into a vein through an access catheter or be used without a distinct tubular access catheter.

CN107625524, US 2005/0276727 and WO2005/062940 disclose different devices to capture relevant particles from a blood flow in a blood vessel. These devices comprise elongated bodies that comprise at their distal ends a receptor surface to capture the particles. These elongated bodies are directly introduced in an access catheter to bring the receptor surface in the blood vessel.

A drawback of these devices is that the insertion and withdrawal of the elongated bodies through the access catheter is susceptible for contamination of the receptor surface with undesired particles.

### SUMMARY OF THE INVENTION

It is an object of the invention to provide an improved medical sampling device for capturing bioanalytes that overcomes one or more of the above-mentioned drawbacks of prior art sampling methods or devices.

The invention provides a medical sampling device for capturing bioanalytes, comprising:
a wire having a receptor part, the receptor part having receptors to bind target bioanalytes,
a channel device defining a channel to receive the wire, the channel having a proximal end and a distal end,
wherein the wire is movable with respect to the channel between at least an extended position in which at least an intravenous section of the receptor part extends from the distal end of the channel and a retracted position in which the intravenous section of the receptor part is retracted into the channel,
wherein the channel device comprises a liquid inlet arranged proximally with respect to at least a part of the intravenous section of the receptor part when the wire is arranged in the retracted position.

This sampling device makes it possible to combine the concepts of in vivo enrichment, hereinafter also referred to as flow biopsy, and solid phase extraction. A flow biopsy is the process of inserting a functionalized wire, i.e. the wire having receptors to bind target bioanalytes, into a bloodstream, where the receptors may selectively bind specific bioanalytes, e.g. circulating tumor cells, extracellular vesicles or bacteria. Flow biopsies are for example useful whenever a bioanalyte is so rare that a blood sample would not provide sufficient bioanalytes for a particular diagnostic procedure. The receptors may be provided in a coating arranged at the receptor part of the wire.

The sampling device of the invention allows to carry out a flow biopsy by arranging the wire of the sampling device in the extended position, such that the intravenous section of the wire may extend out of the channel and into the blood vessel of a patient. The receptors on this intravenous section while present in the blood vessel may bind the bioanalytes of interest, e.g. circulating tumor cells, extracellular vesicles or bacteria. After a certain time period, the intravenous section may be retracted from the blood vessel into the channel by moving the wire from the extended position to the retracted position.

The sampling device may be configured to be inserted at least partially through a venous catheter to place the intravenous section in a blood vessel. As a result, the sampling device does not require a separate catheter of other access port to enter a blood vessel. This prevents additional risk on needle stick injuries and infections. Moreover, the sampling device may be inserted multiple times into the respective blood vessel without the need to create a separate entry into the blood vessel.

Solid phase extraction is a method where a chemical compound is isolated from a mixture. It works by adsorbing the desired compound to a solid (stationary phase), optionally removing any unwanted compounds in one or more washing steps, and ultimately recovering (eluting) the desired compound with a specific solvent. Solid phase extraction is especially useful in biological applications, where it is often applied in the form of immunoaffinity purification.

The sampling device of the invention allows to carry out a solid phase extraction, while the wire is positioned in the retracted position. In this retracted position, liquid that is injected at the liquid inlet may flow through the channel to a distal liquid outlet of the channel, for example a distal end opening through which the wire at least partially extends from the channel when the wire is in the extended position. As the liquid flows from the liquid inlet to the liquid outlet, the liquid flows along the intravenous section of the receptor part, i.e. the section of the receptor part that extended from the channel into a blood vessel when the wire was in the extended position. In this way, a washing liquid may be guided along the receptor part to remove any unwanted compounds in one or more washing steps, and/or an eluting liquid, i.e. a specifically selected solvent, may be guided along the receptor part to recover the desired compound.

The devices of CN107625524, US 2005/0276727 and WO2005/062940 are not configured and not suitable for this combination of in vivo enrichment and solid phase extraction with one sampling device.

The sampling device of the invention further allows for handling of the wire, such as during insertion into a catheter or subsequent retrieval, while the wire is arranged in the retracted position. Doing so greatly reduces the risk of introducing pathogens and other foreign materials, whether from the skin, the catheter, or other elements of the environment, onto the intravenous section of the receptor part of the wire. This in turn greatly reduces the risk of such contamination leading to false positive outcomes and/or misidentification of pathogens in subsequent analyses. In addition, this reduces the risk of introducing such pathogens into the bloodstream and/or catheter where they may cause harm.

In an embodiment, the channel device may be a tube. The tube may be flexible, such that it may be coiled for compactness. The tube may be manufactured by the extrusion of polymer such as PE, PP, POM, PA, PS, PVC, or more preferably of low friction polymers such as PTFE, FEP, PFA and the like. The tube may be an integral element or may be assembled from multiple parts. An internal channel of the insertion tube of an insertion sheath may be regarded to be part of the channel of the medical sampling device when present during the sold phase extraction.

In alternative embodiments, the channel device may be any element or housing defining a channel that is suitable to at least partly accommodate the wire.

In the retracted position of the wire, the liquid inlet is arranged proximally with respect to at least a part of the intravenous section of the receptor part, for example at least 50%, such as 75% of a length of the intravenous section. In an embodiment, the liquid inlet is arranged proximally with respect to 90% of a length of the intravenous section or with its complete length.

The liquid inlet may be embodied as a liquid injection port arranged in the wall of the channel. The liquid injection port may have a commonly used connector for syringes and the like, for example a Luer or Luer-lock. To minimize the contamination risk, a liquid stopper may be provided in the liquid injection port, when the liquid injection port is not in use. The liquid stopper may for example be a male Luer or male Luer-lock cap or plug. Alternatively, the liquid injection port may be provided as a Tuohy Borst or a type of membrane-based barrier. The liquid injection port may be part of the channel device or a separate component mounted on the channel device.

In an alternative embodiment, the liquid inlet may be connected to a deformable reservoir of washing liquid and/or eluting liquid or another liquid that may be introduced into the channel through the liquid inlet by squeezing of the reservoir. Such squeezable reservoir may be fixed to the liquid inlet or may be releasably mounted on the liquid inlet.

In an embodiment, the sampling device comprises an insertion sheath comprising an insertion tube and a connector, wherein the connector is arranged to connect the insertion sheath to the distal end of the channel, wherein the insertion tube is arranged to be inserted into a venous catheter, and wherein the insertion tube comprises an inner channel that when the insertion sheath is connected with the channel device is aligned with the channel to form a conduit.

In this embodiment, the insertion sheath is configured to be introduced into a catheter, for example a standard venous catheter to avoid contact between the receptor part and the venous catheter, when the receptor part is moved to the extended position, thereby reducing the risk of binding of any materials from the catheter on the receptor part. The insertion catheter comprises an insertion tube that can be inserted into a catheter, for example a standard venous catheter. The length of the insertion tube may be selected such that it fully passes the venous catheter. The insertion sheath may for example have a length of 10 to 300 mm, such as 30 to 100 mm.

The insertion sheath therewith reduces the risk of sample contamination and improves sterility during operation of the device. It prevents the picking up of both contaminants from outside the patient and from within the catheter tubes. The insertion sheath may comprise a connector that connects to a connector at the distal end of the channel device, and/or a connector that connects with a venous catheter.

The insertion sheath tube forming the inner channel may be made of a metal such as steel or brass, a polymer such as PU or silicone, for example a low friction polymer PTFE or FEP.

The insertion sheath may be sacrificial, such that it can be discarded after the wire is withdrawn back to the retracted position after the collection of bioanalytes from the bloodstream. Discarding the sacrificial insertion sheath further reduces the contamination risk by ensuring that any contamination on or in the tip of the insertion sheath, such as from the inside of the catheter, cannot come into contact with the elution liquid.

The removal of the insertion sheath also reduces the length of the channel that has to flushed with liquid during solid phase extraction. As a result, the volume of elution liquid to extract the captured bioanalytes from the receptor part may be substantially reduced which results in a higher concentration of the respective bioanalytes in the elution liquid.

In an embodiment, the connecter is configured to provide a releasable connection. The releasable connection can be used to release the insertion sheath from the channel device after the in vivo enrichment has ended.

In an embodiment, the insertion tube has an insertion tube length, wherein the insertion tube length is selected to extend completely through the venous catheter used to introduce the sampling device at least partially into a blood vessel of a patient. When the insertion tube extends completely through the through the venous catheter, the contamination risk of the receptor part with contaminants being present in the venous catheter is substantially reduced.

In an embodiment, the sampling device comprises a venous catheter having a catheter channel. The venous catheter may be provided as a part of the sampling device, but can also be provided as a separate part, for example when using a venous catheter which is already placed in a blood vessel.

In an embodiment, a length of the channel between the liquid inlet and the distal end of the channel is 0.5 to 2 times a length of the intravenous section, for example 1 to 1.5 times the length of the intravenous section. To obtain a relatively high concentration of bioanalytes of interest, it is desirable that the volume of elution liquid that is used to extract the bioanalytes from the intravenous section is relatively small. By having a relative short length of the channel between the liquid inlet and the distal end of the channel, this volume can be kept relatively low.

In an embodiment, the cross section of the receptor part has a non-cylindrical shape. By using a non-cylindrical shape of the cross section of the receptor part in a cylindrically shaped cross section of the inner surfaces of the channel and the insertion tube, the chance of scraping captured particles from the receptor part when moving the wire with respect to the channel may be reduced. In an alternative embodiment, the cross section of the receptor part may be cylindrical and the cross section of the inner surfaces of the channel and the insertion tube may be non-cylindrical. Any other combination of cross section shapes that reduce the risk of scraping off captured particles during movement of the wire with respect to the channel device may also be applied.

In an embodiment, the receptor part comprises guiding surfaces and receptor surfaces, wherein the guiding surfaces are arranged to guide the receptor part along a wall of the channel and wherein at least the receptor surfaces are provided with the receptors to bind target bioanalytes. The guiding surfaces are used to guide the receptor part in the channel. The guiding surfaces may slide along the wall of the channel. The guiding surfaces may advantageously have a relatively small surface area that may come into contact with the wall of the channel to reduce friction forces between channel and the wire.

The guiding surfaces may be provided with receptors, or alternatively, since the guiding surfaces are used to guide the receptor part along the wall of the channel, no receptors may be provided on the guiding surfaces.

The receptor surfaces may be arranged at a smaller radial distance from a longitudinal axis of the receptor part compared to a radial distance of the guiding surfaces from the longitudinal axis, such that the guiding surfaces will contact the wall of the channel, while the receptor surfaces will remain spaced from the wall of the channel. Therewith, the abrasion of the bound bioanalytes from the receptors due to contact between the receptor surfaces and the wall of the channel may be prevented.

The guiding surfaces that are arranged at a larger distance from the longitudinal axis of the receptor part may also facilitate centring of the receptor part in the channel. The centring of the receptor part in the channel has the advantage that there is a uniform and consistent exposure of the receptor surfaces to any injected liquids through the liquid inlet, thereby improving the elution of bound bioanalytes.

The guiding surfaces may extend partially along a cylindrical shape having a diameter that is substantially the same or slightly smaller than the diameter of the channel. By providing guiding surfaces having a shape that at least partially corresponds to the shape of the wall of the channel, the sliding of the receptor part along the wall of the channel may be facilitated, while the receptor part is centred in the channel.

In an embodiment, the cross section of the wire has a central portion about a longitudinal central axis and at least three extensions extending from the central portion. The at least three extensions may advantageously be substantially equally distributed over the circumference of the cross section of the wire, such that the ends of the extensions may be used as guiding surfaces that guide the receptor part along the wall of the channel.

In an embodiment, the cross section of the receptor part may have a substantially polygonal main shape, for example a triangular shape. A polygonal shape may fill a substantial part of the cross section of the channel, therewith creating flow channels between the receptor part and the wall of the channel having a relatively large width extending between the corners of the polygonal shape, and a low height perpendicular to the width. As a result, a relatively small volume of liquid will flow along a relatively large surface width.

In such embodiment, the corners of the polygonal shape may form guiding surfaces, while receptor surfaces are formed by the areas of the circumference extending between the corners. The corners preferably have a relatively small radius such that there is a small contact surface. The small contact surface of the guiding surfaces reduces friction during movement of the wire between the extended and retracted position and prevents the loss of coating and/or any bound bioanalytes by scraping. At the same time a relatively large surface area will be available for the receptor surfaces to bind bioanalytes.

The cross section of the receptor part may be uniform along its length and can be circular or non-circular. In an embodiment, the wire may have a uniform cross section over its entire length.

In an embodiment, a cross section area of the receptor part of the wire fills at least 40% of a cross section area of the channel, for example at least 50% of the cross section area of the channel. By filling a substantial part of the cross section area of the channel with the wire, a relatively small cross section flow area will be used for guiding liquid, e.g. eluting liquid and/or washing liquid from the liquid inlet to the liquid outlet. This has the advantage that there is a relatively small volume of liquid needed to fill and flush the medical sample device with the result that the bioanalytes can be recovered from the medical sample device within a small volume of eluting liquid and therefore with a relatively high concentration.

Further, the relatively small cross section flow surface causes that the elution liquid is passed over a large surface area with relatively high shear force, aiding in the release of the bound bioanalytes and thereby further decreasing the volume of liquid needed to recover the bioanalytes from the receptor part. The high shear force also aids in washing off any undesired and non-specifically bound materials with washing liquid, resulting in improved purity of the sample obtained with the eluting liquid.

In an embodiment, the receptor part is provided at least partially with a coating comprising the receptors. The coating applied to at least a part of the surface of the receptor part of the wire is used for the selective capture of bioanalytes from the bloodstream, and can in addition be used for imparting hemocompatibility, reduction of friction during manipulation, and reducing fouling. The coating comprises receptors, which specifically bind the bioanalytes. The coating may additionally comprise a linker, which covalently attach to the receptor part and receptors, improving the steric availability of the receptors. The linker may also impart or improve the hemocompatibility of the wire. A cleavable linker may provide a gentle and selective method to retrieve the bioanalytes after capture from the bloodstream.

The coating may comprise one or more receptors that specifically bind bioanalytes through affinity interaction, charge interaction or other means. The receptor may be a protein such as an antibody, antibody fragment, lectin, or other proteins or a small molecule, aptamer, or other molecule. The receptors may be directly covalently attached to the receptor part, or to a linker molecule that is attached to the receptor part of the wire. The receptor may be attached through a covalent bond or non-covalent interactions such as streptavidin-biotin or protein A/G-antibody.

The linker may impart or improve the hemocompatibility of the substrate, reduce the friction during manipulation, attach the receptor to the functionalized wire, and may provide a cleavable site for the gentle and selective release of captured bioanalytes. The linker may be attached to the functionalized wire surface directly, or may be attached after providing a functional group to the surface such as amines, carboxylic acids and thiols, or may be attached to the surface through one or more intermediate linkers such as bifunctional molecules or silanes. This coating process of the linker and or receptor may be carried out using processes such as dip-coating.

One advantage of using a linker as described above is that it allows more attachment sites for receptors that bind bioanalytes to the surface of the functionalized wire. Suitable binding sites on the surface of the receptor part may only bind one receptor, whereas if the same site has a linker bound to it, and that linker has multiple binding sites for receptors, then the amount of available receptors per unit of surface area on the receptor part is increased, which in turn increases the capture effectiveness for the target bioanalyte. Another advantage of using a linker is that it can project the receptors away from the surface of the receptor part and into the bloodstream, therefore increasing the effectiveness of bioanalyte capture. A further advantage of using a linker as described above is that the linker can be cleaved enzymatically or chemically. This cleaving ensures that the captured bioanalyte is gently removed from the surface of the receptor part without damaging or otherwise affecting the bioanalyte itself. It can then be used in further downstream processing and diagnostic steps. While cleaving the linker to remove captured bioanalytes from the receptor part may be a desirable step for some diagnostic techniques, certain analyses and diagnostics of the captured bioanalytes can be carried out with the bioanalytes still bound to the surface of the receptor part.

The coating may have a non-fouling component that minimizes the non-specific binding, pick-up or capture of blood components or other materials other than the target bioanalyte. The non-fouling component may comprise the cleavable linker that is used as the attachment site for receptors, in the case that the linker also exhibits the necessary non-fouling characteristics. If the cleavable linker does not present suitable non-fouling characteristics, it may be used in combination with a linker that does exhibit these non-fouling characteristics, but that is not suitable as an attachment site for receptors. Non-fouling linkers consist of, but are not limited to, polysaccharides, PEG, poly-amino acids, amino acids, or zwitterionic molecules or polymers.

An advantage of using non-fouling components is that non-specific binding of blood components other than the target bioanalyte is reduced, thereby making sample purification in downstream processing and diagnostics steps simpler and more effective. A further advantage of using non-fouling components is that binding sites on the functionalised wire that might otherwise be occupied by blood components that are not the target bioanalyte, are free to be occupied by the target bioanalyte, increasing yield and further improving the effectiveness of downstream processing and diagnostics.

In an embodiment, the wire comprises a stiff core wire and at least at the receptor part a polymer material provided on the core wire.

The wire may be formed partially or fully of a material that can be provided with a coating. Advantageously, the wire comprises, at least at the receptor part a polymer to which a covalently-bound coating may be provided. The polymer may contain ester bonds within its polymer chain, which can be conveniently cleaved by aminolysis, which is advantageous because many coatings may be conveniently applied to resultant surface amino groups. Polymeric materials that contain ester bonds in their chain include, but are not limited to, polyesters, co-polyesters, polyurethanes, polycarbonates, polymethylmethacrylates and certain liquid crystal polymers. The polymer can be hemocompatible.

To increase the stiffness of the wire a relatively stiff core wire may be provided in the wire. The core wire can be at the receptor part embedded within the polymer, wrapped or coiled around the polymer.

The core wire may be advantageously made of a material that is suitable for remaining in the bloodstream for an extended period, including, but not limited to, glass fibre, carbon fibre, carbon nanotubes, or metals such as magnesium, tantalum, niobium, cobalt, chromium, nickel, iron, or alloys of these. The core wire is advantageously made of a material that has good “pushability” and “torquability” to make it suitable for navigating through tubing, including, but not limited to, materials such as nitinol or stainless steel. When a core wire is used, the cross sectional shape of the receptor part can be imparted by the shape of the core wire and/or by the shape of the polymer material provided on the core wire.

The wire may be manufactured using extrusion, co-extrusion, injection moulding, over-moulding, casting, dip-coating, laser-cutting, or ultrasonic welding.

The polymeric material may be sufficiently stiff and mechanically strong to be capable of being deployed the required distance through the catheter and into the bloodstream, and then retracted again, without buckling, stretching, or otherwise deforming the wire.

In an embodiment, the wire extends proximally from a proximal end of the channel. The part of the wire that extends proximally from the proximal end of the channel may be used to manipulate the wire, in particular to move the wire between the retracted position and the extended position. A stop element may be mounted on the wire or the channel device to prevent the wire to be moved beyond a maximal insertion depth, i.e. a maximal extension beyond the distal end of the channel. The stop element may be mounted on the wire and have an outer diameter greater than the inner diameter of the channel.

In an embodiment the length of the channel between the liquid inlet and the proximal end of the channel is more than the total translation of the wire between the retracted position and the extended position, such that any potential contaminant on the wire may not be introduced beyond the liquid inlet, thereby preventing contamination of the sample.

In another embodiment, the wire is, in its retracted position completely housed within a closed space. This means that the wire does not extend proximally from the channel into the environment. Such embodiment enables a fully enclosed upstream section of the device, which is advantageous for keeping the device contaminant free.

The wire may for example be coiled on a reel that can be rotated to move the wire between a retracted position and an extended position. Translation of the wire may for example be created by gears or wheels in contact with the wire at opposite sides thereof. An advantage of this embodiment is that the translation is not predetermined, which allows for compatibility of the device with a wide range of venous catheters with varying lengths. The combination of a coiled reel in a closed housing results in a compact device with low risk of contamination.

In an embodiment, the sampling device comprises one or more stops to block movement of the wire from the retracted position in proximal direction of the channel and/or to block movement of the wire from the extended position in distal direction of the channel. It may be desirable that movement of the wire with respect to the channel in proximal direction is limited to prevent that the receptor part is moved in proximal direction past the liquid inlet. This ensures that all liquid that flows from the liquid inlet to the liquid outlet will flow at least along a part of the length of the intravenous section of the receptor part. Also, it may be desirable that movement of the wire with respect to the channel in distal direction is limited to prevent that the receptor part is moved in distal direction too far into a patient.

The stop may for example be formed by a stop surface of the wire having an increased thickness that is not able to pass a stop opening with decreased width in the channel. The increased thickness of the wire and/or decreased width in the channel may extend over the complete circumference or a part thereof. For example, the cross section of the part with increased thickness on the wire may be circular, while the stop opening with decreased width in the channel is triangular and will not let the circular part with increased thickness through. Also other shape may be used, such as an increased diameter of the wire that cannot pass a smaller diameter of the channel.

In an embodiment, the sampling device comprises a seal arranged proximally of the liquid inlet, at least when the wire is in the retracted position, wherein the seal prevents liquid flow through the channel. It is desirable that the liquid injected through the liquid inlet will flow along the receptor part to a liquid outlet distally from the receptor part in retracted position. To ensure that most liquid flows in distal direction from the liquid inlet, a seal is provided proximally from the liquid inlet that blocks liquid flow in proximal direction, at least when the wire is in the retracted position. The seal should block the passage of liquid and/or air, while still allowing for the movement of the wire between the retracted position and the extended position.

The seal may for example be a thickened part on the wire, that contacts the wall of the channel to provide a sealing engagement between the wire and the wall. In another embodiment, the seal may be formed by a sealing ring, or another opening, arranged in the channel that is in sealing engagement with the wire.

The seal may for example be made of a flexible material like silicone or TPU that for example may be overmolded or welded or otherwise made onto the wire.

In an embodiment, the seal may also function as a stop that blocks movement of the wire from the retracted position in proximal direction of the channel.

In an embodiment, a lubricant is provided in the channel to reduce friction between the wire and the wall of the channel. This also facilitates controlled movement of the wire between the retracted position and the extended position.

In an embodiment, a storage solution is provided in the channel. The storage solution may lubricate the wire during the insertion and withdrawal procedure, may prevent the coating and its constituent parts from degrading over time, and/or may reduce the risk of introducing air bubbles into the bloodstream. The storage solution may be contained within the channel and may be inserted along with the wire into the bloodstream. The storage solution may also need to be flushed or replaced prior to insertion.

The storage solution may include solutions of glycerol, polyethylene glycol, sugars such as sucrose, glucose and/or trehalose; polyols such as mannitol, sorbitol, and/or inositol; surfactants such as tween 20 and/or albumin; amino acids such as histidine, glycine and/or arginine; salts such as sodium chloride. The storage solution may be selected such that the pH and ionic strength improve the stability of the coating. By carefully selecting the makeup of the solution that the wire is stored in, the coating will remain stable for a prolonged period of time. The composition of the storage solution may also be optimized for lubrication, for example by the addition of polymers like polyethylene glycol or polysaccharides like heparin and hyaluronic acid. In summary, by carefully selecting the makeup of the solution that the functionalized wire is stored in, the coating will remain stable for a prolonged period of time and the insertion procedure may be improved by reduced friction.

The invention further provides the use of the sampling device of any of the claims 1-16 to carry out a flow biopsy, comprising the steps of:
moving the wire in longitudinal direction of the channel from the retracted position to the extended position such that at least an intravenous section of the receptor part extends from the distal end of the channel into a blood vessel, and
after a certain time period moving the wire back to the retracted position in which the intravenous section of the receptor part is retracted into the channel.

In an embodiment, the use comprises before moving the wire from the retracted position to the extended position connecting an insertion sheath having an insertion tube to the distal end of the channel and inserting the insertion tube into a catheter, for example a standard venous catheter, inserted into a blood vessel of a patient. The length of the insertion tube may be selected such that it fully passes the insertion catheter. The insertion sheath may for example be connected to the tube as a preparation step for use of the medical sampling device or during assembly of the medical sampling device in a production/assembly facility. Assembly during a preparation step has the advantage that the selection of the insertion tube may be adapted to the catheter in use, while connecting the insertion sheath in a production/assembly facility may reduce the risk of contamination in the connection step.

In an embodiment, the use comprises after moving the wire back to the retracted position, retracting the insertion tube from the catheter and/or disconnecting the insertion tube from the distal end of the channel device.

The invention further provides the use of the sampling device of any of the claims 1-24 to carry out a solid phase extraction, while the wire is in the retracted position, comprising the steps of:
injecting an eluting liquid into the liquid inlet to recover a desired compound from the receptors, and
collecting the eluting fluid with compound in a collector, for example a vial.

In an embodiment, the use comprises the step of injecting a washing liquid into the liquid inlet to remove any unwanted compounds in one or more washing steps.

### BRIEF DESCRIPTION OF THE DRAWINGS

FIG. 1A show a first embodiment of a medical sampling device according to the invention in a storage position;

FIG. 1B shows the embodiment of FIG. 1A introduced into an intravenous catheter;

FIG. 1C shows the embodiment of FIG. 1A wherein the receptor part extends from the distal end of the channel, including an enlarged detail of the surface of the receptor part;

FIG. 1D shows the embodiment of FIG. 1A after the elution procedure;

FIG. 2 shows a cross section of the receptor part of the embodiment of FIGS. 1A-1D arranged in the channel;

FIGS. 3A and 3B show two alternative embodiments of a cross section of the receptor part; and

FIG. 4 shows a second embodiment of a medical sampling device according to the invention.

### DETAILED DESCRIPTION

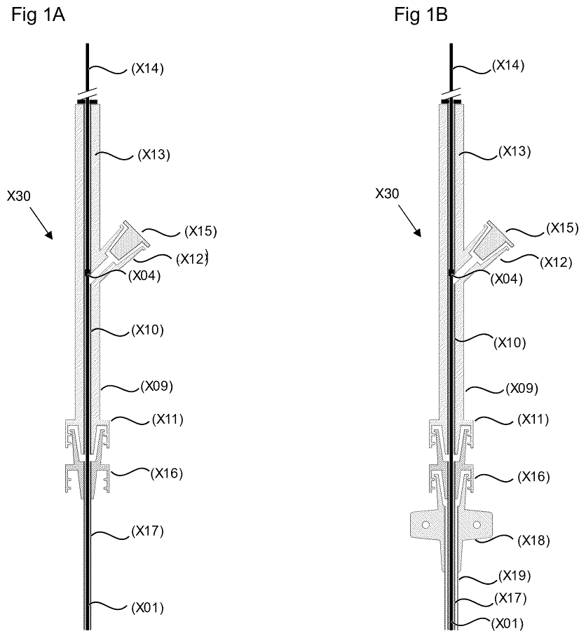

FIGS. 1A-1D show a first embodiment of a medical sampling device for capturing bioanalytes, generally denoted by reference numeral (X30). The medical sampling device (X30) comprises a wire (X14) having a receptor part (X01) at its distal end and a channel device in the form of a tube (X13) having a channel (X10).

The wire (X14) extends through the channel (X10) and is movable in longitudinal direction of the tube with respect to the tube (X13) between at least an extended position (FIG. 1C) in which an intravenous section of the receptor part (X01) to be inserted into a blood vessel extends from the distal end of the tube (X13) and a retracted position (FIG. 1D) in which the intravenous section of the receptor part (X01) is retracted into the channel (X10). The wire extends proximally from the proximal end of the tube to allow manipulation of the wire, in particular to move the wire between the retracted position and the extended position.

The overall length of the wire (X14) may be between 5 cm and 90 cm, for example between 20 cm and 80 cm, for instance between 30 cm and 60 cm. The length of the intravenous section of the wire (X14) used for sampling purposes is therefore between approximately 2 cm and 45 cm, more specifically between 3 cm and 30 cm and more specifically between 5 cm and 15 cm.

The tube (X13) is made of a low friction polymer such as PTFE, FEP, PFA and the like. In the tube (X13) a liquid injection port (X12) is provided that can be used to inject a liquid into the channel (X10). The liquid injection port (X12) comprises a Luer-lock to connect a syringe (X22) to the liquid injection port (X12). When the liquid injection port (X12) is not in use to inject liquid, a Luer cap (X15) can be placed on the Luer lock of the liquid injection port (X12) to close the liquid injection port (X12).

It is desirable that liquid injected through the liquid injection port (X12) into the channel (X10) will flow towards the distal end (X09) of the channel (X10). Therefore, a thickened seal element (X04) is arranged on the wire (X14) that is made of a flexible sealing material, such as silicone, and is in sealing contact with the wall of the channel (X10).

At the distal end (X09) of the tube a connector (X11) is provided to connect the tube (X13) to a connector of an insertion sheath (X16). The insertion sheath (X16) comprises an insertion tube (X17) comprising an inner channel. When the insertion sheath (X16) is connected to the tube (X13) the inner channel of the insertion tube is aligned with the channel (X10) to form a conduit through which the wire (X14) extends.

The insertion sheath (X16) is configured to be introduced into a catheter, for example a standard venous catheter (X18) to avoid contact between the receptor part (X01) of the wire (X14) and the venous catheter (X18). The length of the insertion tube (X17) is selected to extend completely through the venous catheter (X18) to avoid any contact between the wire (X14), in particular its receptor part (X01) and the venous catheter (X18).

The insertion sheath (X16) comprises a second connector, such as a Luer connector, to connect the insertion sheath (X16) with a corresponding connector of the venous catheter (X18).

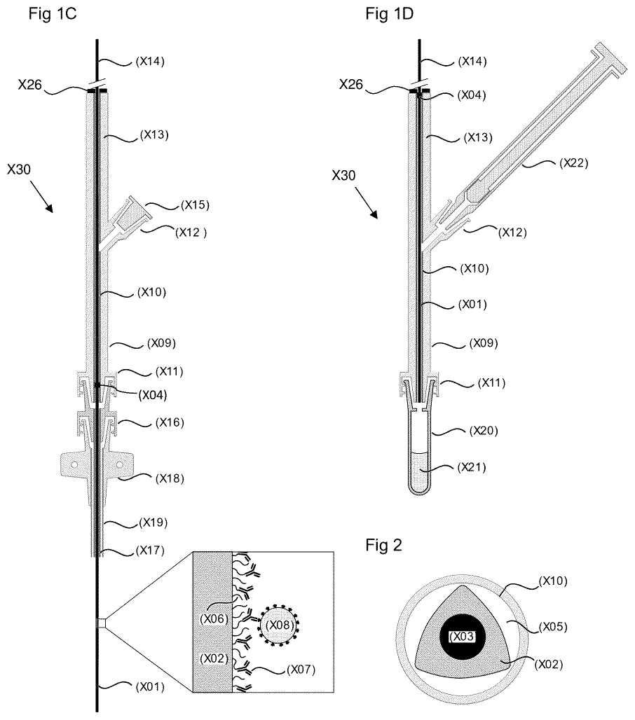

FIG. 2 shows a cross section of the wire (X14) at the receptor part (X01). The wire (X14) comprises a metal core wire (X03) embedded in a polymeric material (X02). On the polymeric material (X02) a coating is provided. The coating comprises receptors (X07; FIG. 1C) to bind target bioanalytes (X08). The coating may additionally comprise a linker (X06, FIG. 1C), which covalently attach to the polymeric material (X02) and receptors (X07), improving the steric availability of the receptors (X07).

The metal core wire (X03) is made of a material that has good “pushability” and “torquability” to make it suitable for navigating through tubing, including, but not limited to, materials such as nitinol or stainless steel. The core wire (X03) has a circular cross section, but the polymeric material defines a non-circular cross section of the receptor part (X01).

The cross section of the receptor part (X01) is triangular in shape. The triangular shape defines three corners that form guiding surfaces, i.e. surfaces that are used to guide the receptor part (X01) along the channel (X10) and/or the inner channel of the insertion tube (X17).

The surface areas between the corners form receptor surfaces provided with the coating comprising receptors (X07) to bind target bioanalytes (X08), and linker (X06). Between the receptor surfaces and the wall of the channel (X10) flow channels (X05) are formed that allow the flow of liquid injected through the channel (X10).

The triangular shape of the cross section is dimensioned such that a radial distance of the corners from a longitudinal axis of the wire (X14) is slightly smaller than the radius of the channel (X10). The corners are therefore located close to the wall of the channel (X10). This shape ensures a centring of the receptor part (X01) in the channel (X10) with three flow channels (X05), each having the same cross section. This centring of the receptor part (X01) has the advantage that there is a uniform and consistent exposure of the receptor surfaces to any injected liquids through the liquid injection port (X12), thereby improving the elution of bound bioanalytes. Furthermore, the shape of the flow channels (X05) as created by the triangular shape of the receptor part (X01) provides a flow having a cross section with a relatively wide side extending along the receptor surfaces and a relatively low height, i.e. a small distance between the receptor surfaces and the wall of the channel (X10). As a result, a small volume of liquid flows along a relatively large receptor surface width.

The corners of the triangular shape may be flattened to correspond to the shape of the wall of the channel (X10), facilitating the sliding of the receptor part (X01) with its guiding surfaces along the wall of the channel (X10).

As shown in FIG. 2, the cross section area of the receptor part (X01) fills at least 40% of the cross section area of the channel (X10), for example at least 50% of the cross section area of the channel (X10). By filling a substantial part of the cross section area of the channel (X10) with the receptor part (X01) of the wire (X13), the flow channels (X05) will take in a relatively small cross section area for guiding liquid. This has the advantage that there is a relatively small volume of liquid needed to fill and flush the medical sample device with the result that the bioanalytes can be recovered from the medical sample device with a small volume of eluting liquid and therefore with a relatively high concentration.

The sampling device (X30) makes it possible to combine the concepts of flow biopsy and solid phase extraction.

FIG. 1A shows the combination of a medical sampling device (X30) comprising the tube (X13), the wire (X14) and the insertion sheath (X16), as described above.

FIG. 1B shows the insertion tube (X17) of the insertion sheath (X16) arranged into a venous catheter (X18), whereby the insertion sheath is connected with its connector to the connector of the venous catheter (X18). The venous catheter (X18) may be arranged in a blood vessel of a patient (not shown). The insertion tube (X17) fully extends through the venous catheter (X18) and therefore the wire (X14) does not come into contact with the venous catheter (X18). When the wire (X14) is moved to the extended position, an intravenous section of the receptor part (X01) extends into the blood vessel of the patient, where bioanalytes (X08) may bind to the receptors (X07) coated on the receptor part (X01) of the wire (X14).

FIG. 1C shows the intravenous section of the receptor part (X01) that extends out of the tube and insertion tube into the blood vessel.

After that the intravenous section remained within the blood vessel for a desired period of time, the wire (X14) can be moved to the retracted position shown in FIG. 1D, for example by pulling the wire (X14) extending proximally from the proximal end of the tube (X13) in the proximal direction. A stop element (X26) that cooperates with sealing element (X04) may be provided to prevent movement of the wire (X14) past the retracted position.

When the wire (X14) is positioned in the retracted position as shown in FIG. 1D, the medical sampling device (X30) may be pulled backwards to pull the insertion tube (X17) of the insertion sheath (X16) out of the venous catheter (X18). Subsequently or alternatively, the tube (X13) and wire (X14) may be dismounted from the insertion sheath (X16) and the connector (X11) at the distal end (X09) of the tube (X13) may be mounted on a vial (X20)

In FIG. 1D, the medical sampling device (X30) is shown mounted on the vial (X20), and a syringe (X22) with liquid is mounted on the liquid injection port (X12)

The medical sampling device (X30) now allows to carry out a solid phase extraction, while the intravenous section of the receptor part (X01) is positioned in the retracted position. In this retracted position, the intravenous section is positioned distally of the liquid injection port (X12). Liquid that is injected through the liquid injection port (X12) will flow through the channel (X10) to a distal liquid outlet of the channel, and into the vial (X20). As the liquid flows from the liquid injection port (X12) to the vial, the liquid flows along the intravenous section of the receptor part, i.e. the section of the receptor part that extended from the tube into the blood vessel when the wire was in the extended position. In this way a washing liquid may be guided along the receptor part to remove any unwanted compounds in one or more washing steps, and an eluting liquid, i.e. a specifically selected solvent, may be guided along the receptor part to recover the desired compound.

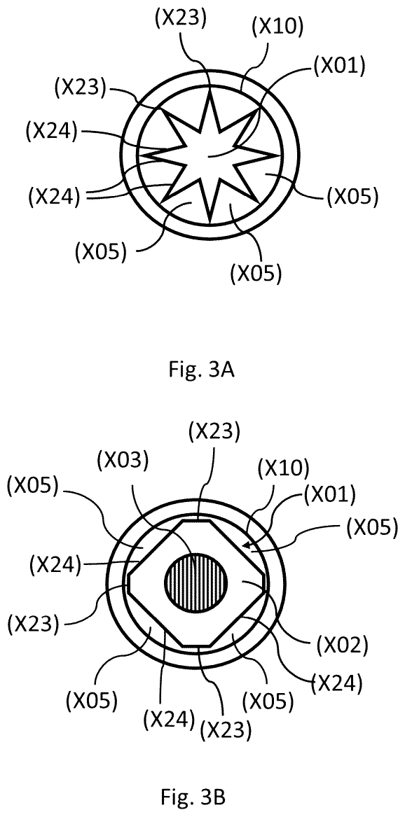

FIGS. 3A and 3B show two alternative embodiments of a cross section of the receptor part (X01) arranged in the channel (X10).

In the embodiment of FIG. 3A, the cross section of the receptor part (X01) is star shaped having eight points. The ends of the eight points form guiding surfaces (X23) to centre and guide the receptor part (X01) in the channel (X10). The side surfaces of the points form receptor surfaces (X24) provided with a coating comprising receptors (X07) to bind target bioanalytes (X08). The guiding surfaces (X23) may also be provided with this coating, but since these guiding surfaces (X23) will be moved along the wall of the channel (X10), these guiding surfaces (X23) are less suitable to hold the bioanalytes (X08) of interest due to the abrasive forces between the guiding surfaces (X23) and the wall of the channel (X10). Between the receptor surfaces (X24) and the wall of the channel (X10) flow channels (X05) are formed that allow the flow of liquid injected to flow through the channel (X10) along the receptor surfaces (X24). Due to the star shape of the cross section of the receptor part (X10), the surface area of the receptor surfaces (X24) is relatively large compared to the cross section of the flow channels (X05).

The cross section of the receptor part (X01) is made of a single material, for example a polymer material.

In the embodiment of FIG. 3B, the cross section of the receptor part (X01) is square with bevelled corners. The four bevelled corners form guiding surfaces (X23) to centre and guide the receptor part (X01) in the channel (X10). The four main side surfaces of the square form receptor surfaces (X24) provided with a coating comprising receptors (X07) to bind target bioanalytes (X08). The guiding surfaces (X23) may also be provided with this coating. Similarly to the embodiments of FIGS. 2 and 3A, flow channels (X05) are formed between the receptor surfaces (X24) and the wall of the channel (X10) that allow the flow of liquid injected to flow through the channel (X10) along the receptor surfaces (X24). Also in this embodiment, the surface area of the receptor surfaces (X24) is relatively large compared to the cross section of the flow channels (X05). The cross section of the receptor part (X01) is made of a metal core wire (X03) and a polymer material (X02) arranged on the core wire (X03).

It will be clear for the skilled person that other cross sections may also be contemplated.

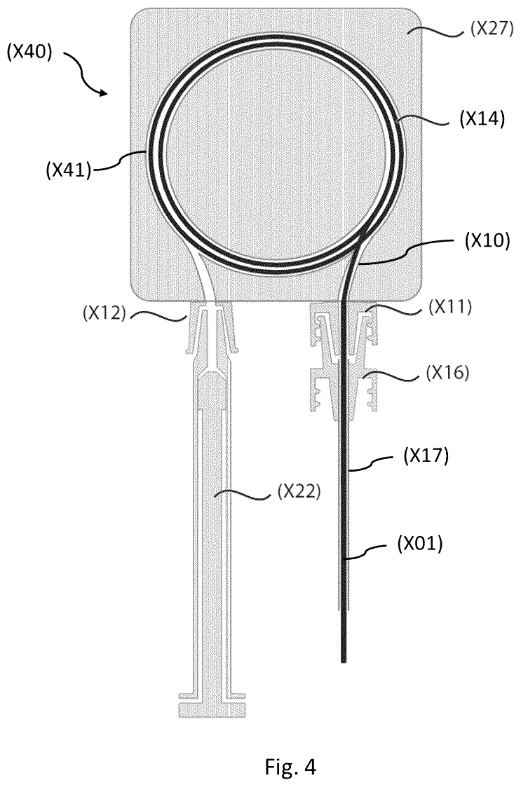

FIG. 4 shows an alternative embodiment of a medical sampling device (X40) having a channel device in the form of a closed housing (X27) defining a channel (X10) having a proximal end at a liquid injection port (X12) and a distal end at distal connector (X11). At the distal connector (X11) an insertion sheath (X16) having an insertion tube (X17) is connected. The insertion tube (X17) can be introduced into a catheter inserted into a blood vessel of a patient, as described with respect to the embodiment of FIG. 1A-1D.

In the channel (X10) a wire (X14) is provided having a receptor part (X01) at its distal end comprising receptors (X07) to bind target bioanalytes (X08). The wire (X14) is movable with respect to the channel (X10) between an extended position in which an intravenous section of the receptor part (X01) extends from the medical sampling device (X40) and a retracted position in which the receptor part is retracted into the channel (X10).

The channel (X10) has a circular part (X41) in which the wire (X14) is partly coiled starting from the proximal end of the wire (X14). As a result, in the retracted position of the wire (X14), the wire (X14) will be completely arranged within the closed housing (X27). This will substantially reduce the risk of entering of contaminations in the channel (X10). The movement of the wire may for example be created by a gear (not shown) acting on the wire, or a rotatable part of the closed housing (X27) may be rotatable with respect to the rest of the housing and the proximal end of the wire (X27) may be connected to this rotatable part to allow the wire to be moved between the retracted position and the extended position.

A syringe (X22) can be connected to the liquid injection port (X12) to inject washing liquid and/or eluting liquid into the channel (X10) to carry out a solid phase extraction.

1. A medical sampling device for capturing bioanalytes, comprising:
a wire having a receptor part, the receptor part having receptors to bind target bioanalytes,
a channel device defining a channel to receive the wire, the channel having a proximal end and a distal end,
wherein the wire is movable with respect to the channel between at least an extended position in which at least an intravenous section of the receptor part extends from the distal end of the channel and a retracted position in which the intravenous section of the receptor part is retracted into the channel,
wherein the channel device comprises a liquid inlet arranged proximally with respect to at least a part of the intravenous section of the receptor part when the wire is arranged in the retracted position.

2. The sampling device of claim 1, wherein the sampling device comprises an insertion sheath comprising an insertion tube and a connector, wherein the connector is arranged to connect the insertion sheath to the distal end of the channel, wherein the insertion tube is arranged to be inserted into a venous catheter, and wherein the insertion tube comprises an inner channel that when the insertion sheath is connected with the channel device is aligned with the channel to form a conduit.

3. The sampling device of claim 2, wherein the connecter is configured to provide a releasable connection.

4. The sampling device of claim 2, wherein the insertion tube has an insertion tube length, wherein the insertion tube length is selected to extend completely through the venous catheter used to introduce the sampling device at least partially into a blood vessel of a patient.

5. The sampling device of claim 2, wherein the insertion tube has an insertion tube length, wherein the insertion tube length is 10 to 300 mm, for example 30 to 100 mm.

6. The sampling device of claim 2, wherein the sampling device comprises a venous catheter having a catheter channel.

7. The sampling device of claim 6, wherein the insertion tube fully extends through the catheter channel.

8. The sampling device of claim 1, wherein a length of the channel between the liquid inlet and the distal end of the channel is 0.5 to 2 times a length of the intravenous section, for example 1 to 1.5 times the length of the intravenous section.

9. The sampling device of claim 1, wherein the cross section of the receptor part has a non-cylindrical shape.

10. The sampling device of claim 1, wherein the receptor part comprises guiding surfaces and receptor surfaces, wherein the guiding surfaces are arranged to guide the receptor part along a wall of the channel and wherein at least the receptor surfaces are provided with the receptors to bind target bioanalytes.

11. The sampling device of claim 10, wherein the guiding surfaces extend partially along a cylindrical shape having substantially a diameter that is substantially the same or slightly smaller than the diameter of the channel.

12. The sampling device of claim 1, wherein the cross section of the receptor part has a central portion about a longitudinal axis and at least three prongs extending from the central portion.

13. The sampling device of claim 1, wherein the cross section of the wire has a central portion about a longitudinal axis and at least three prongs extending from the central portion.

14. The sampling device of claim 1, wherein the cross section of the receptor part has a substantially polygonal main shape.

15. The sampling device of claim 1, wherein a cross section area of the receptor part of the wire fills at least 40% of a cross section area of the channel.

16. The sampling device of claim 1, wherein the receptor part is provided at least partially with a coating comprising the receptors.

17. The sampling device of claim 1, wherein the wire comprises a stiff core wire and at least at the receptor part a polymer material provided on the core wire.

18. The sampling device of the preceding claim 17, wherein the core wire is made of a metal.

19. The sampling device of claim 1, wherein the wire extends proximally from a proximal end of the channel.

20. The sampling device of claim 1, wherein the length of the channel between the liquid inlet and the proximal end of the channel is more than the total translation of the wire between the retracted position and the extended position.

21. The sampling device of claim 1, wherein the wire is, in its retracted position completely housed within a closed space.

22. The sampling device of claim 1, wherein the sampling device comprises one or more stops to block movement of the wire from the retracted position in proximal direction of the channel and/or to block movement of the wire from the extended position in distal direction of the channel.

23. The sampling device of claim 22, wherein the stop comprises a stop surface of the wire and a stop opening in the channel, wherein the stop surface of the wire has an increased thickness that is not able to pass the stop opening in the channel that has a decreased width.

24. The sampling device of claim 1, wherein the sampling device comprises a seal arranged proximal of the liquid inlet, at least when the wire is in the retracted position, wherein the seal prevents at the seal liquid flow through the channel.

25. Use of A method of using the sampling device of claim 1, to carry out a flow biopsy, comprising the steps of:
moving the wire with respect to the channel from the retracted position to the extended position such that at least an intravenous section of the receptor part extends from the distal end of the channel into a blood vessel, and
after a certain time period moving the wire back to the retracted position in which the intravenous section of the receptor part is retracted into the channel.

26. A method of using the sampling device of claim 1 to carry out a solid phase extraction, while the wire is in the retracted position, comprising the steps of:
injecting an eluting liquid into the liquid inlet to recover a desired compound from the receptors, and
collecting the eluting fluid with compound in a collector.
